(19) United States
(12) Patent Application Publication
(10) Pub. No.: US 2014/0017299 A1
(43) Pub. Date: Jan. 16, 2014

(54) STEROID HORMONE DELIVERY SYSTEMS AND METHODS OF PREPARING THE SAME

(71) Applicant: MONOSOL RX, LLC, (US)

(72) Inventors: Eric Dadey, Furlong, PA (US); Garry L. Myers, Kingsport, TN (US); Alexander Mark Schobel, Whitehouse Station, NJ (US)

(73) Assignee: MONOSOL RX, LLC, Warren, NJ (US)

(21) Appl. No.: 13/843,933

(22) Filed: Mar. 15, 2013

Related U.S. Application Data
(63) Continuation-in-part of application No. 13/588,731, filed on Aug. 17, 2012.
(60) Provisional application No. 61/524,847, filed on Aug. 18, 2011.

(51) Int. Cl. A61K 9/127; A61K 31/568

(52) U.S. Cl. CPC A61K 9/127; A61K 31/568
USPC 424/450; 514/170; 424/400

(57) ABSTRACT

The present invention is directed to film dosage compositions for delivery of testosterone esters. In particular, a film dosage composition is provided that includes: a) a first region including: i) a first polymeric matrix and ii) a first plurality of particles including: 1) a first testosterone ester and 2) a first surfactant; and b) a second region including: i) a second polymeric matrix and ii) a second plurality of particles including: 1) a second testosterone ester and 2) a second surfactant.

### CROSS-REFERENCE TO RELATED APPLICATION

This application in a continuation-in-part of U.S. patent application Ser. No. 13/588,731 filed on Aug. 17, 2012 which claims the benefit of priority to U.S. Provisional Patent Application Ser. No. 61/524,847 filed on Aug. 18, 2011. Both of the aforementioned applications are incorporated by reference herein in their entirety for all purposes.

### FIELD OF THE INVENTION

The present invention provides steroid hormone delivery systems and methods of preparing the same. In particular, the steroid hormone delivery systems provided include a primary construct including one or more hydrophobic steroid hormone esters in the form of a liposome, a lipid particle, a micelle, an emulsion, a nanoparticle, or a niosome wherein the primary construct is formulated into a secondary construct for administration. In one embodiment, the secondary construct is in the form of a film suitable for administration to a mucosal surface, e.g., oral, vaginal, rectal, nasal or ocular surfaces. Particularly useful mucosal surfaces are the buccal, lingual, and sublingual surfaces. In another embodiment, the secondary construct is in the form of a liquid suspension suitable for enteral and/or parenteral administration.

### BACKGROUND OF THE RELATED TECHNOLOGY

There is a need for delivery systems to administer steroid hormones for medicinal indications with favorable pharmacokinetics that foster increased patient compliance and/or provide increased patient comfort during administration thereof. For example, non-injectable formulations for sustained release of steroid hormones are desirable. There is a particular need for delivery systems for steroid hormones which can achieve approximate steady state levels of hormones in the blood relative to prior delivery methods.

### SUMMARY OF THE INVENTION

The present invention provides steroid hormone delivery systems and methods of preparing the same which overcome the problems associated with prior delivery systems. In particular, steroid hormone delivery systems are provided having a primary construct with one or more hydrophobic steroid hormone esters in a liposome, a lipid particle, a micelle, an emulsion, a nanoparticle, or a niosome wherein the primary construct is formulated into a secondary construct having at least one pharmaceutically acceptable excipient. The secondary construct desirably is in the form of a solid dosage or semi-solid form or a liquid dosage form, such as a suspension. Additionally, the present invention provides methods of forming a steroid hormone depot.

Advantageously, such delivery systems exploit the hydrophobic nature of steroid hormones and, in essence, provide a delivery system within a delivery system which has favorable pharmacokinetics upon administration thereof.

The present invention also provides film dosage compositions for delivery of testosterone esters. In particular, a film dosage composition is provided that includes: a) a first region including: i) a first polymeric matrix and ii) a first plurality of particles including: 1) a first testosterone ester and 2) a first surfactant; and b) a second region including: i) a second polymeric matrix and ii) a second plurality of particles including: 1) a second testosterone ester and 2) a second surfactant. In one embodiment, the first region is a first film and the second region is a second film.

### BRIEF DESCRIPTION OF THE FIGURES

FIG. 1 shows a graph of plasma testosterone levels over a 12 hr period in three minipigs following buccal administration of both 11.11 mg testosterone enanthate (TE) film and 12.67 mg testosterone undecanoate (TU) film.

FIG. 2 shows a graph of plasma testosterone levels over a period of time in three minipigs following topical administration of 20 mg FORSTESTA® (testosterone gel).

FIG. 3 shows a graph of plasma testosterone levels over a 12 hr period in three minipigs following buccal administration of a bilayer film dosage composition of the present invention containing 13.89 mg testosterone enanthate (TE) film and 15.84 mg testosterone undecanoate (TU).

FIG. 4 shows a graph of plasma testosterone levels over a period of time in three minipigs following topical administration of 20 mg FORSTESTA® (testosterone gel).

FIG. 5 shows a graph of combining the data from FIGS. 3 and 4 for purposes of comparison.

### DETAILED DESCRIPTION OF THE INVENTION

As used herein, the term “construct” means a delivery system for release of active. In the context of the present invention, a “primary” construct refers to a delivery system which is formulated using one or more hydrophobic steroid hormones wherein the primary construct itself is used as a “component” in formulating a “secondary” construct which further includes at least one pharmaceutically acceptable excipient. In other words, a delivery system containing active is itself used as a component to formulate a higher order delivery system.

In one aspect, the present invention provides steroid hormone delivery systems including: a primary construct having one or more hydrophobic steroid hormones and one or more of the following: a lipid, an oil, a polymer, or a surfactant; wherein the primary construct is in the form of a micelle, a liposome, a lipid particle, an emulsion, a nanoparticle, or a niosome; and a secondary construct including the primary construct and at least one pharmaceutically acceptable excipient.

In one embodiment, the primary construct includes a surfactant and the primary construct is in the form of a micelle. In one embodiment, the primary construct includes a polymer and the primary construct is in the form of a micelle. In one embodiment, the primary construct includes a lipid and the primary construct is in the form of a liposome. In one embodiment, the primary construct includes both a lipid and a surfactant, and the primary construct is in the form of a lipid particle. In one embodiment, the primary construct includes both a surfactant and oil, and the primary construct is in the form of an emulsion. In one embodiment, the primary construct includes a surfactant and the primary construct is in the form of a niosome.

In one embodiment, the secondary construct is in the form of a liquid suspension. In one embodiment, the secondary construct is in the form of a film. In one embodiment, the secondary construct is in the form of a liquid dosage form, solid dosage form or semisolid dosage form.

In another aspect, the present invention provides methods of preparing a steroid hormone delivery system including: preparing a primary construct having one or more hydrophobic steroid hormones and one or more of the following: a lipid, an oil, a polymer, or a surfactant; wherein the primary construct is in the form of a micelle, a liposome, a lipid particle, an emulsion, a nanoparticle, or a niosome; and preparing a secondary construct wherein the primary construct and at least one pharmaceutically acceptable excipient is formulated into a dosage form for administration.

In yet another aspect, the present invention provides methods of forming a steroid hormone depot wherein a liquid suspension of the present invention is administered parenterally.

In still yet another aspect, the present invention provides methods of forming a steroid hormone depot wherein a film of the present invention is administered sublingually, lingually, buccally, vaginally or rectally.

In some embodiments, the delivery systems of the present invention provide release of steroid hormone for at least 3 hours to about 4 hours, at least 4 hours to about 8 hours, at least 8 hours to about 12 hours, at least 12 hours to about 24 hours, at least 12 hours or at least 24 hours. In one embodiment, the delivery systems provide sustained release of steroid hormone for 1 to 7 days, greater than 7 days, at least 10 days, at least 14 days, at least 21, or at least 30 days.

Suitable hydrophobic steroid hormones include, but are not limited to, testosterone esters for delivery of testosterone. In particular, suitable testosterone esters include, but are not limited to, testosterone enanthate, testosterone cypionate, testosterone undecanoate, testosterone propionate, testosterone formate, testosterone acetate, testosterone butyrate, testosterone valerate, testosterone caproate, testosterone isocaproate, testosterone heptanoate, testosterone octanoate, testosterone nonanoate, testosterone decanoate or a combination of two or more thereof.

Suitable lipids for the preparation of liposomes include, but are not limited to, cholesterol, cholesterol sulfate, phosphatidic acid, phosphatidylcholine, phosphatidylethanolamine, phosphatidylglycerol, phosphatidylserine, lysophosphatidylcholine, phosphatidylinositol, phosphatidylinositol phosphate, phosphatidylinositol bisphosphate, phosphatidylinositol triphosphate, ceramide phosphorylcholine, ceramide phosphorylethanolamine, ceramide phosphorylglycerol or a combination of two or more thereof.

The polymer may be water soluble, water swellable, water insoluble, or a combination of one or more either water soluble, water swellable or water insoluble polymers.

Specific examples of useful water soluble polymers include, but are not limited to, polyethylene oxide (PEO), pullulan, hydroxypropylmethyl cellulose (HPMC), hydroxyethyl cellulose (HPC), hydroxypropyl cellulose, polyvinyl pyrrolidone, carboxymethyl cellulose, polyvinyl alcohol, sodium aginate, polyethylene glycol, xanthan gum, tragancanth gum, guar gum, acacia gum, arabic gum, polyacrylic acid, methylmethacrylate copolymer, carboxyvinyl copolymers, starch, gelatin, and combinations thereof. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(a-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of a-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°-347° F. (170°-175° C.); lactide/glycolide 100 L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

In embodiments, polyethylene oxide (PEO), when used alone or in combination with a hydrophilic cellulosic polymer, achieves flexible, strong films. Additional plasticizers or polyalcohols are not needed for flexibility. Non-limiting examples of suitable cellulosic polymers for combination with PEO include HPC and HPMC. PEO and HPC have essentially no gelation temperature, while HPMC has a gelation temperature of 58-64° C. (Methocel EF available from Dow Chemical Co.). Moreover, these films are sufficiently flexible even when substantially free of organic solvents, which may be removed without compromising film properties. As such, if there is no solvent present, then there is no plasticizer in the films. PEO based films also exhibit good resistance to tearing, little or no curling, and fast dissolution rates when the polymer component contains appropriate levels of PEO.

To achieve the desired film properties, the level and/or molecular weight of PEO in the polymer component may be varied. Modifying the PEO content affects properties such as tear resistance, dissolution rate, and adhesion tendencies. Thus, one method for controlling film properties is to modify the PEO content. For instance, in some embodiments rapid dissolving films are desirable. By modifying the content of the polymer component, the desired dissolution characteristics can be achieved.

In accordance with the present invention, PEO desirably ranges from about 20% to 100% by weight in the polymer component. In some embodiments, the amount of PEO desirably ranges from about 1 mg to about 200 mg. The hydrophilic cellulosic polymer ranges from about 0% to about 80% by weight, or in a ratio of up to about 4:1 with the PEO, and desirably in a ratio of about 1:1.

In some embodiments, it may be desirable to vary the PEO levels to promote certain film properties. To obtain films with high tear resistance and fast dissolution rates, levels of about 50% or greater of PEO in the polymer component are desirable. To achieve adhesion prevention, i.e., preventing the film from adhering to the roof of the mouth, PEO levels of about 20% to 75% are desirable. In some embodiments, however, adhesion to the roof of the mouth may be desired, such as for administration to animals or children. In such cases, higher levels of PEO may be employed. More specifically, structural integrity and dissolution of the film can be controlled such that the film can adhere to mucosa and be readily removed, or adhere more firmly and be difficult to remove, depending on the intended use.

The molecular weight of the PEO may also be varied. High molecular weight PEO, such as about 4 million, may be desired to increase mucoadhesivity of the film. More desirably, the molecular weight may range from about 100,000 to 900,000, more desirably from about 100,000 to 600,000, and most desirably from about 100,000 to 300,000. In some embodiments, it may be desirable to combine high molecular weight (600,000 to 900,000) with low molecular weight (100,000 to 300,000) PEOs in the polymer component.

For instance, certain film properties, such as fast dissolution rates and high tear resistance, may be attained by combining small amounts of high molecular weight PEOs with larger amounts of lower molecular weight PEOs. Desirably, such compositions contain about 60% or greater levels of the lower molecular weight PEO in the PEO-blend polymer component.

To balance the properties of adhesion prevention, fast dissolution rate, and good tear resistance, desirable film compositions may include about 50% to 75% low molecular weight PEO, optionally combined with a small amount of a higher molecular weight PEO, with the remainder of the polymer component containing a hydrophilic cellulosic polymer (HPC or HPMC).

Suitable polymeric compounds for the preparation of polymeric micelles include, but are not limited to, polymeric compounds from the following classes of polymeric compounds: poly(ethylene oxide)-b-poly(propylene oxide)s, poly(ethylene oxide)-b-poly(ester)s, and poly(ethylene oxide)-b-poly(amino acid)s. Also contemplated is the use of a combination of two or more polymeric compounds from either the same polymeric class or a different polymeric class listed above.

Suitable surfactants for the preparation of micelles include, but are not limited to, sodium dodecylsulfate, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monooleate, sorbitan monostearate, sorbitan palmitate, sorbitan monolaurate or a combination of two or more thereof.

Suitable oils for the preparation of emulsions include, but are not limited to, liquid paraffin, vegetable oil, olive oil, avocado oil, almond oil, castor oil, sesame oil, jojoba oil, wheatgerm oil, sunflower oil, mineral oil, isopropyl myristate, or a combination of two or more thereof.

Emulsions (e.g., microemulsions and nanoemulsions) include one or more surfactants and one or more oils. Additionally, one or more co-surfactants may optionally be employed. Suitable components for preparing emulsions include, but art not limited to, one or more surfactants, one or more oils and one or more optional co-surfactants listed below in Table 1.

TABLE 1

| SURFACTANT | OIL | COSURFACTANT |
|---|---|---|
| Sodium dodecylsulfate | Liquid Paraffin, | 1-Butanol |
| Polyoxyethylene (20) | Vegetable Oil | 1-Pentanol |
| sorbitan monolaurate | | |
| Polyoxyethylene (20) | Olive Oil, | Diethyleneglycol |
| sorbitan monopalmitate | | monoethyl ether |
| Polyoxyethylene (20) | Almond Oil, | |
| sorbitan monostearate | | |
| Polyoxyethylene (20) | Avocado Oil, | |
| sorbitan monooleate | | |
| Sorbitan monooleate | Jojoba Oil, | |
| Sorbitan monostearate | Wheatgerm Oil, c | |
| Sorbitan palmitate | Castor Oil, | |
| Sorbitan monolaurate | Sunflower Oil | |
| | Sesame Oil | |
| | Mineral Oil | |
| | Isopropyl Myristate | |

Lipid particles include one or more lipids and one or more surfactants. Suitable components for the preparation of lipid particles include, but are not limited to, one or more lipids and one or more surfactants listed below in Table 2.

TABLE 2

| LIPID | SURFACTANT |
|---|---|
| Glyceryl Monosterate | Poloxamer 188 |
| Glyceryl Distearate | Poloxamer 407 |
| Stearic Acid | |
| Glyceryl Behanate | |

Suitable routes of administration of the drug delivery system include, but are not limited to, oral, buccal, sublingual, lingual, parenteral, intravenous, intramuscular, subcutaneous, transdermal, intraperitoneal, intraocular, nasal, inhalational, topical, vaginal or rectal.

Suitable dosage forms include, but are not limited to, liquid dosage forms, solid dosage forms and semisolid dosage forms. In one embodiment, the secondary construct is in the form of a liquid dosage form, solid dosage form or semisolid dosage form.

Exemplary suitable dosage forms include films, pills, tablets, capsules, liquid suspensions (e.g., for oral, ocular, nasal or inhalatory administration or for parenteral injection), flakes, powders, creams, suppositories, and transdermal patches.

Exemplary methods of preparing film delivery systems are described in U.S. Pat. Nos. 7,357,891, 7,897,080, 7,666,337, 7,824,588 and 7,910,031 and Published U.S. Patent Application Nos. US 2011/00033542 and US 2011/00033541, the contents of each of which are incorporated herein by reference in their entirety. Additionally, exemplary methods of preparing pharmaceutical dosage forms are described in Remington: The Science and Practice of Pharmacy. 21st Edition. Philadelphia, Pa. Lippincott Williams & Wilkins (2005), the contents of which are incorporated herein by reference in its entirety. Notably, suitable pharmaceutically acceptable excipients depend on the dosage form being prepared and are also described in Remington: The Science and Practice of Pharmacy (supra).

In some embodiments of the present invention, the film dosage compositions are multilayer films. In some embodiments, the two or more film layers that form the multi-layer film are compositionally the same. Each film layer contains the same polymer composition and any optional ingredients. In other embodiments, the two or more film layers may be different. The layers may compositionally differ in any manner, such as, different polymers, actives, flavors or other optional ingredients.

For example, a film that effervesces when placed in the mouth may be provided by incorporating an edible acid into one film layer or film pocket and a base into the other film layer or film pocket. When the film is consumed, the saliva causes the film to dissolve and the acid and base react to produce effervescence. Alternatively, the acid and base may be separated by a coating and present in a single layer. Suitable edible acids include, but are not limited to, citric acid, phosphoric acid, tartaric acid, malic acid, ascorbic acid and combinations thereof. Suitable bases include, but are not limited to, alkali metal carbonates, alkali metal bicarbonates, alkaline earth metal carbonates, alkaline earth metal bicarbonates and combinations thereof.

The layers also may differ physically, such as different sizes, shapes or thicknesses. For example, the film layers may be round, square or rectangular. Film layers of different thicknesses may be used to create a controlled release multi-layer film.

As described above, the multi-layer films include two or more film layers that may be the same or different. In some bi-layer embodiments, the film layers are in full face-to-face engagement with each other. In some embodiments, the multi-layer film has more than two layers, such as three-layer film.

In other embodiments of the present invention, the film layers are in partial face-to-face engagement with each other. The partial face-to-face engagement may be perimetric to the film. The film layers may be joined, or laminated, at the perimetric engagement.

In accordance with the present invention, the film layers may be joined at the point of their at least partial face-to-face engagement. The film layers may be joined in any manner known to those skilled in the art. For instance, the film layers may be laminated together using heat and/or pressure to seal the layers. The incorporation of a polymer having a low glass transition temperature is desirable for heat sealing the film layers together as it softens at a low temperature.

Alternatively, the film layers may be adhesively or solvent bonded together independent of the glass transition temperature of the polymer composition.

The film layers may be sealed in any shape, such as squared or rounded edges, among others. In some embodiments, the point of engagement, i.e., the fusion or sealing area, is judiciously chosen to be minimized as such lamination creates a greater film thickness and potentially slower dissolution time. Additionally, bunching and/or densification of film may occur, particularly in certain shapes, such as sharp-edged shapes, which may be slower dissolving at those lamination areas. As such, rounded edges may be desired in some embodiments to limit the amount of lamination area and speed the dissolution time and rate. Dissolution time, of course, also is related to the compositional and physical characteristics of the film, the solvent medium, the actives used, and the temperature at which the film is being dissolved, among others.

Though not meant to be limited by any theory with the subject invention, it is anticipated that, when the drug delivery system of the present invention is applied to the sublingual, lingual, or buccal mucosal surface, the primary construct will absorb into the mucosal tissue and will release the steroid hormone in the aqueous environment of the mucosal tissue. The first construct provides a stabilized form of the hormone and permits incorporation of the hormone into an appropriate second construct, e.g., a delivery system or dosage form, such as a film, which further permits travel across the mucosal membrane. In the case of buccal administration for example, a film may carry the first construct and preferentially release the first construct into and through the mucosa of the buccal tissue. The deposition of the first construct into the tissue may form a type of reservoir or depot in the tissue. Desirably, the first construct preferentially falls apart, or dissociates the hormone, e.g., precipitates out of the first construct into the surrounding tissue, allowing for the slow and continued release into the bloodstream. This release coupled with the hydrophobic nature of the steroid hormone is believed to result in the deposition of the steroid hormone as a “solid” substance in the mucosal tissue. This depot of steroid hormone is then slowly dissolved and absorbed into the systemic circulation. It is believed that steady-state plasma levels of steroid hormone are achievable for at least several hours including at least 24 hours following administration of a single dose. Likewise, it is believed that parenteral administration of the drug delivery system of the present invention is similarly conducive for depot formation.

The features and advantages of the present invention are more fully shown by the following examples which are provided for purposes of illustration, and are not to be construed as limiting the invention in any way.

### EXAMPLES

### Primary Construct

The primary construct includes one or more hydrophobic steroid hormone esters in a liposome, a lipid particle, a micelle, an emulsion, a nanoparticle, or a niosome. Although testosterone ester is exemplified in the following primary constructs, other hydrophobic steroid hormone esters may supplement or be substituted for testosterone ester.

### Micelles

One procedure for preparing a micellar formulation of one or more testosterone esters is to first suspend a known amount of one or more testosterone esters in a volume of water with constant stifling. A solution of surfactant and/or polymeric material is prepared in water. Small aliquots of the surfactant and/or polymeric solution are added periodically to the suspension of testosterone ester(s) with constant stifling. The resulting solution is inspected after each addition and subsequent aliquots are added just until all of the testosterone ester(s) dissolve as indicated by the solution being visually clear.

Alternatively, the components are reversed but result in the formation of a similar micellar formulation. Specifically, a solution is prepared by dissolving a known amount of surfactant and/or polymer in water. Small aliquots of powder testosterone ester(s) are added to the solution with constant stirring to produce a visually clear solution. Additional aliquots of powder testosterone ester are added periodically until the solution exhibits a permanent cloudy, opalescent, or turbid appearance.

Using either of the aforementioned procedures, the optimum ratio of surfactant and/or polymer to testosterone ester(s) is that where the maximum amount of testosterone ester(s) is solubilized by the minimum amount of surfactant and/or polymer.

### Liposomes

In general, liposomal formulations of therapeutics agents are prepared in a multi-step procedure. In the first step, known amounts of lipids (e.g., cholesterol), phospholipids (e.g., phosphatidylcholine, phosphatidylethanolamine) and testosterone ester(s) are dissolved in ethanol. The resulting solution is added to a round-bottom flask and ethanol removed by rotary evaporation. This process results in deposition of the lipids, phospholipids and testosterone ester(s) as a thin layer coating the inside surface of the round bottom flask.

The lipid/testosterone layer is then hydrated with water alone or an aqueous solution containing any combination of salts, pH modifiers, preservatives (e.g., antimicrobial agents), or other stabilizing additives. This hydration process produces liposomal vesicles of various sizes (e.g., diameters approaching 10 to 20 μm) each containing an aqueous core with testosterone ester intercalated into the lipid bilayer.

Mechanical stress (e.g., sonication, extrusion, microfluidization) is then applied to the liposome-containing testosterone suspension in order to reduce and generate liposomes of uniform size.

### Lipid Particles

Lipid particles containing testosterone ester(s) can be prepared by using an emulsification technique. According to this method, a mixture of lipid (e.g., glycerol behenate), surfactant (e.g., poloxamer 407) and testosterone ester(s) is heated to an elevated temperature sufficient to melt the oil and dissolve the testosterone ester(s). Water heated to the same temperature as the oil/surfactant/testosterone ester(s) mixture is added slowly and the resulting dispersion emulsified using a mixer operating at several thousand rpms. The emulsion produced is cooled to room temperature with constant stifling until solidification resulting in the production of lipid microparticles.

### Niosomes

The preparation of niosomes-containing testosterone esters is comparable to the preparation of liposomes. The primary difference is that niosomes use synthetic, non-ionic surfactants (e.g., dialkyl polyglycerol ethers) rather than naturally derived phospholipids to form the lipid bilayer of the vesicles. Otherwise, similar methods are employed. In particular, components are dissolved in a solvent, the solvent is evaporated and the dried components are hydrated followed by exposure to mechanical stress can be used to form niosome-containing testosterone esters of uniform size.

### Emulsions

Emulsions containing testosterone ester(s) can be prepared by dispersing one liquid into another. For example, emulsions can be prepared by mixing, at several thousand rpms, testosterone ester(s), one or more surfactants, one or more oils, water and optionally one or more co-surfactants. Microemulsions and nanoemulsions refer to the size of the particles dispersed therein.

Tables 3 and 4 provide specific examples (formulation compositions) of films that contain a testosterone ester formulated as a microemulsion within the film. The example in Table 3 uses testosterone enanthate as the testosterone ester, whereas the three examples detailed in Tables 4A, 4B and 4C, respectively, use testosterone undecanoate as the testosterone ester. These types of microemulsions are often referred to in the scientific literature as self-emulsfying drug delivery systems (SEDDS).

TABLE 3
11.11 mg Testosterone Enanthate (C111) Formulation*

| | 11.11 mg Testosterone Enanthate |
|---|---|
| | (C111) Formulation with |
| Component | Etocas 35/Transcutol HP/Capryol 90 |
| HPMC | 25.3110% (11.390 mg) |
| PEO | 12.6555% (5.695 mg) |
| Maltitol added as Lycasin | 12.6555% (5.695 mg) |
| 80/55 Roquette | |
| Testosterone Enanthate (C111) | 24.6890% (11.110 mg) |
| Etocas 35 NF (Cremophor EL) | 10.6160% (4.777 mg) |
| Transcutol HP | 1.7290% (0.778 mg) |
| Caprylol 90 | 12.3440% (5.555 mg) |
| % Solids | 30 |
| % Moisture | 1.38 |
| Dry Target Strip Weight | 45 mg |
| Target Strip Weight to Account for | 45.63 mg |
| % Moisture | |
| Strip Size | 13 × 22 mm |

*11.11 mg Testosterone enanthate (C111) is equivalent to 8 mg testosterone base

TABLE 4A
12.67 mg Testosterone Undecanoate (C111) Film Formulations*

| | 12.67 mg Testosterone Undecanoate |
|---|---|
| | Formulation Using Capryol 90/Gelucire |
| Component | 50/13 System |
| HPMC | 31.214% (23.410 mg) |
| PEO | 15.607% (11.705 mg) |
| Sucralose | 2.000% (1.500 mg) |
| Peceol | 0.500% (0.375 mg) |
| Testosterone Undecanoate | 16.893% (12.670 mg) |
| Gelucire 50/13 | 16.893% (12.670 mg) |
| Capryol 90 | 16.893% (12.670 mg) |
| % Solids | 30 |
| % Moisture | 1.57 |
| Dry Target Strip Weight | 75 mg |
| Target Strip Weight to Account | 76.196 mg |
| for Moisture Content | |
| Strip Weight Range | 74 to 83 mg |
| Strip Size | 22 × 20 mm |

*12.67 mg Testosterone Undecanoate (C111) is equivalent to 8 mg Testosterone base

TABLE 4B
12.67 mg Testosterone Undecanoate (C111) Film Formulations*

| | 12.67 mg Testosterone Undecanoate |
|---|---|
| | Formulation Using Lauroglycol 90/ |
| Component | Gelucire 50/13 System |
| HPMC | 31.214% (23.410 mg) |
| PEO | 15.607% (11.705 mg) |
| Sucralose | 2.000% (1.500 mg) |
| Peceol | 0.500% (0.375 mg) |
| Testosterone Undecanoate | 16.893% (12.670 mg) |
| Gelucire 50/13 | 16.893% (12.670 mg) |
| Lauroglycol 90 | 16.893% (12.670 mg) |
| % Solids | 30 |
| % Moisture | 0.81 |
| Dry Target Strip Weight | 75 mg |
| Target Strip Weight to Account for | 75.612 mg |
| Moisture Content | |
| Strip Weight Range | 72 to 80 mg |
| Strip Size | 22 × 20 mm |

*12.67 mg Testosterone Undecanoate (C111) is equivalent to 8 mg Testosterone base

TABLE 4C
12.67 mg Testosterone Undecanoate (C111) Film Formulations*

| | 12.67 mg Testosterone Undecanoate |
|---|---|
| Component | Formulation Using Gelucire 50/13 System |
| HPMC | 31.214% (23.410 mg) |
| PEO | 15.607% (11.705 mg) |
| Sucralose | 2.000% (1.500 mg) |
| Peceol | 0.500% (0.375 mg) |
| Testosterone Undecanoate | 16.893% (12.670 mg) |
| Gelucire 50/13 | 33.786% (25.340 mg) |
| % Solids | 25 |
| % Moisture | 0.78 |
| Dry Target Strip Weight | 75 mg |
| Target Strip Weight to | 75.590 mg |
| Account for Moisture Content | |
| Strip Weight Range | 72 to 81 mg |
| Strip Size | 22 × 20 mm |

*12.67 mg Testosterone Undecanoate (C111) is equivalent to 8 mg Testosterone base

As part of the preclinical evaluation of these testosterone ester formulations, the pharmacokinetic profile of the testosterone enanthate prototype identified in Table 3 and the testosterone undecanoate identified in Table 4C were compared to the pharmacokinetic profile of FORTESTA® (testosterone gel) in minipigs.

Briefly, on Day 1, three (3) castrated Gottingen minipigs were anesthetized, the oral cavity was exposed and the enanthate film was placed on the buccal mucosa and the undecanoate film was placed on the opposite buccal surface of each pig. That is, each pig had two films applied to the oral mucosa. Each film was formulated with a nominal testosterone dose of 8 mg; therefore, the total dose that each pig received was 16 mg testosterone.

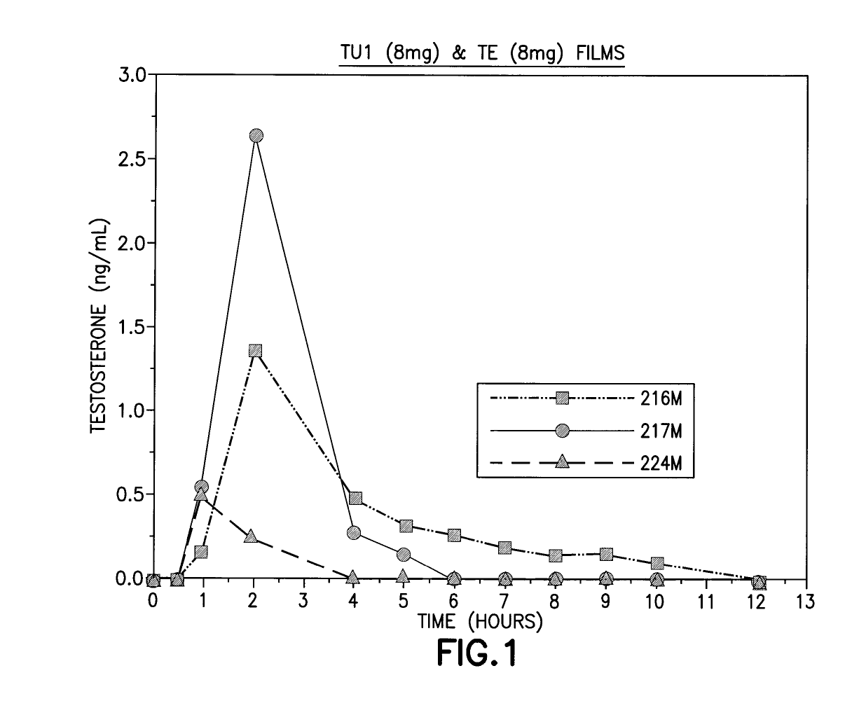

Blood samples were collected periodically over 12 hours and the plasma analyzed for testosterone using an HPLC-MS/MS analytical method. The pharmacokinetic profile of each pig is shown in FIG. 1.

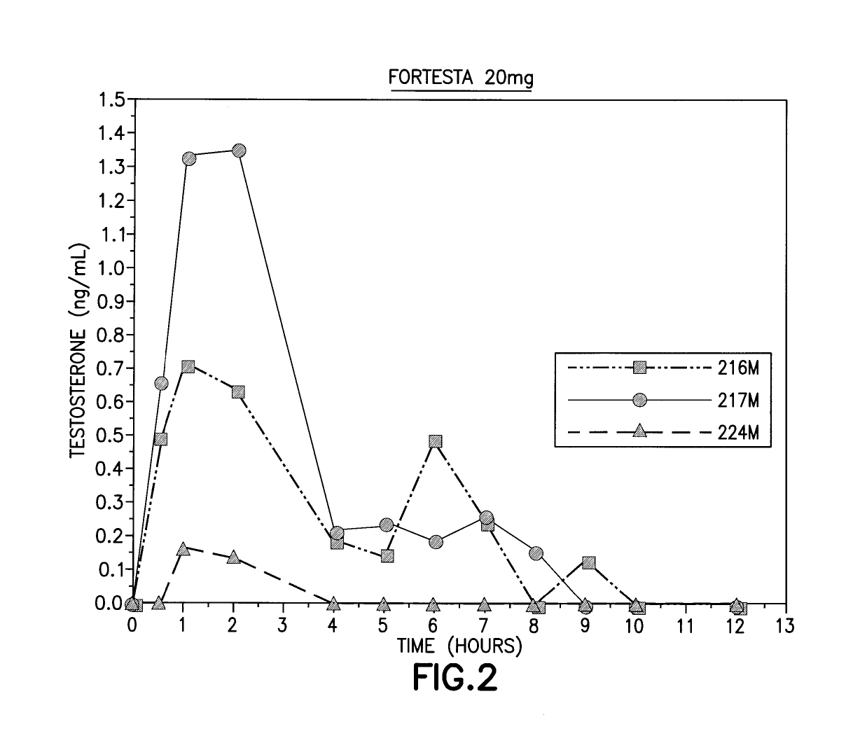

In a control study, the same three minipigs were dosed with 20 mg FORTESTA® (testosterone) topical gel. Blood samples were also collected periodically over 12 hours from each animal and the plasma analyzed for testosterone using an HPLC-MS/MS analytical method. The pharmacokinetic profile of each pig is shown in FIG. 2.

A comparison of the pharmacokinetic profiles of the buccal Testosterone ester films (FIG. 1) to the pharmacokinetic profiles of topical FORTESTA® (testosterone) gel (FIG. 2) shows that both dosage forms provide sustained delivery of testosterone for a minimum of 8 hours. In fact, in Animal Number 216M, the buccal films provide detectable levels of testosterone for at least 10 hours post dosing, whereas no animal in the FORTESTA® (testosterone gel) group showed detectable testosterone levels beyond 10 hours.

Of equal importance is the fact that the total exposure to the drug substance is lower for the buccal films (16 mg) as compared to the topical gel (20 mg). Taken together, the results suggest that the buccal films may provide a therapeutic effect similar to FORTESTA® (testosterone gel) using a lower overall dose.

### Bilayer Film Dosage Composition

A film dosage composition of the present invention was prepared as follows:

### Preparation of 13.89 mg Testosterone Enanthate (TE) (10 mg Base) Formulation

A film containing testosterone enanthate was prepared as follows:

### Preparation of Polymer Solution

The weight of the small fabricated glass bowl and stirrer was obtained to allow QS with water later.

The following ingredients were added to the small fabricated glass bowl (all percentages listed are percentages of solids in the solution except where designated otherwise):

### a) 17.50 g of Distilled Water

### b) 0.037 g (0.50%) Peceol

### c) 0.0002 g (0.002%) FD & C red #40

A blend of the below ingredients was then added to the fabricated glass bowl and stirred with a spatula for a short time:

### c) 3.398 g (45.302%) HPMC E15 (Methocel E15 Premium LV)

### d) 1.699 g (22.648%) Polyethylene Oxide (PEO) WSR N80 LEO

### e) 0.150 g (2.000%) Sucralose

The solution was prepared as described below using the Degussa Dental Multivac Compact:

| | | |
|---|---|---|
| 40 Minutes | Stirring = 125 rpm | Vacuum = 60% (18.5 in Hg) |
| 40 Minutes | Stirring = 125 rpm | Vacuum = 90% (26 in Hg) |
| 20 Minutes | Stirring = 125 rpm | Vacuum = 95% (27 in Hg) |
| 12 Minutes | Stirring = 125 rpm | Vacuum = 98% (28 in Hg) |
| 4 Minutes | Stirring = 125 rpm | Vacuum = 100% (29 in Hg) |

Added distilled water to obtain QS

| | | |
|---|---|---|
| 4 Minutes | Stirring = 125 rpm | Vacuum = 100% (29 in Hg) |

Thus, a polymer solution was prepared with a solids content of 30% and a run size of 25 grams.

### Preparation of TE/Surfactant Solution

1.385 g Testosterone Enanthate (TE) and 1.385 g surfactant solution which is composed of 43% Etocas 35, 7% Transcutol HP, and 50% Capryol 90 were added to a screw cap vial (these percentages are percentages of surfactant solution rather than percentages of solids). The contents of the vial, which is composed of the TE/surfactant solution, were heated in an 80° C. oven to obtain a clear solution.

### Addition of the TE/Surfactant Solution to the Polymer Solution and Preparation of Film

The vial containing the TE/surfactant solution and the plastic dropper were zeroed on a balance to allow addition of the correct amount of the TE/surfactant solution by difference.

2.216 g of the TE/urfactant Solution which contains 1.108 g (14.774%) TE and 1.108 g (14.774%) surfactants were added to the bowl containing the polymer solution as quickly as possible while stirring vigorously with a spatula.

The stirrer was then added to the bowl and stirred with vacuum for 20 minutes to deaerate the solution and to more efficiently mix the contents. A final vacuum of 100% was obtained to insure good deaeration. This was achieved by slowly reducing vacuum on the following schedule: 4 minutes at 60%, 4 minutes at 90%, 4 minutes at 95%, 4 minutes at 100%, QS with water, and 4 more minutes at 100%.

The final solution was cast into wet film using a K-Control Coater with the micrometer wedge bar height at 750 microns. The film was allowed to dry for 24 minutes in an 80° C. convection air oven. The film was cut into 22 by 25 mm strips to obtain strips with a dry target weight of 94 mg.

### Preparation of a 15.84 mg Testosterone Undecanoate (TU) (10 mg Base) Formulation

A film containing testosterone undecanoate was prepared as follows:

### Preparation of Polymer Solution

The weight of the small fabricated glass bowl and stirrer was obtained to allow QS with water later.

The following ingredients were added to the small fabricated glass bowl (all percentages are percentages of solids in the solution):

a) 0.031 g (0.50%) Peceol

b) 0.0001 g (0.002%) FD & C Blue #1 Granular

c) 18.75 g Distilled Water

A blend of the following ingredients was then added to the fabricated glass bowl and stirred with a spatula for a short time:

### d) 1.951 g (31.213%) HPMC E15 (Methocel E15 Premium LV)

### e) 0.975 g (15.606%) PEO WSR N80 LEO

### f) 0.125 g (2.00%) Sucralose

The solution was prepared as described below using the Degussa Dental Multivac Compact:

| | | |
|---|---|---|
| 40 Minutes | Stirring = 125 rpm | Vacuum = 60% (18.5 in Hg) |
| 40 Minutes | Stirring = 125 rpm | Vacuum = 90% (26 in Hg) |
| 20 Minutes | Stirring = 125 rpm | Vacuum = 95% (27 in Hg) |
| 12 Minutes | Stirring = 125 rpm | Vacuum = 98% (28 in Hg) |
| 4 Minutes | Stirring = 125 rpm | Vacuum = 100% (29 in Hg) |

Added distilled water to obtain QS

| | | |
|---|---|---|
| 4 Minutes | Stirring = 125 rpm | Vacuum = 100% (29 in Hg) |

Thus, a polymer solution was prepared with a solids content of 25% and a run size of 25 grams.

### Preparation of TU/Gelucire 50/13 Solution

1.32 g TU and 2.64 g Gelucire 50/13 were added to a screw cap vial. The contents of the vial were heated in an 80° C. oven to obtain a clear solution.

### Addition of the TU/Gelucire 50/13 Solution to the Polymer Solution and Preparation of Film

The polymer solution in the bowl with the stirrer top was heated in an 80° C. oven while the TU/Gelucire 50/13 Solution was heating. The polymer solution was placed in a Styrofoam insulator to help keep the bowl and contents warm while adding active solution.

The vial containing the TU/Gelucire 50/13 solution and the plastic dropper were zeroed on a balance to allow addition of the correct amount of the TU/Gelucire 50/13 Solution by difference.

3.168 g of the TU/Gelucire 50/13 solution which contains 1.056 g (16.893%) TU and 2.112 g (33.786%) Gelucire 50/13 were added to the heated bowl containing the polymer solution as quickly as possible while stifling vigorously with a spatula. The TU/Gelucire 50/13 solution remained melted throughout the addition. After the addition was complete, distilled water was added to obtain QS.

The stirrer was then added to the bowl and stirred with vacuum for 20 minutes to deaerate the solution and to more efficiently mix the contents. A final vacuum of 100% was obtained to insure good deaeration. This was achieved by slowly reducing vacuum on the following schedule: 4 minutes at 60%, 4 minutes at 90%, 4 minutes at 95%, 4 minutes at 100%, QS with water, and 4 more minutes at 100%.

The final solution was cast into wet film using a K-Control Coater with the micrometer wedge bar height set 900 microns. The film was allowed to dry for 26 minutes in an 80° C. convection air oven. The film was cut into 22 by 25 mm strips to obtain strips with a dry target strip weight of 93.75 mg.

The film strips had the following make-up:

TABLE 5
Composition of TE and TU Film Strips (10 mg Base)

| | 13.89 mg | 15.84 mg |
|---|---|---|
| | TE Film Strips | TU Film Strips |
| HPMC | 45.302% (42.583 mg) | 31.213% (29.262 mg) |
| PEO | 22.648% (21.289 mg) | 15.606% (14.631 mg) |
| Sucralose | 2.000% (1.880 mg) | 2.000% (1.875 mg) |
| Peceol | 0.500% (0.470 mg) | 0.500% (0.469 mg) |
| Testosterone Enanthate | 14.774% (13.888 mg) | None |
| Red # 40 Colorant | 0.002% (0.002 mg) | None |
| FD & C Blue # 1 | None | 0.002% (0.002 mg) |
| Granular | | |
| Testosterone | None | 16.893% (15.837 mg) |
| Undecanoate | | |
| Gelucire 50/13 | None | 33.786% (31.674 mg) |
| Etocas 35 | 6.353% (5.972 mg) | None |
| Transcutol HP | 1.034% (0.972 mg) | None |
| Capryol 90 | 7.387% (6.944 mg) | None |
| % Solids | 30 | 25 |
| Dry Target Strip Weight | 94 mg | 93.75 mg |
| Strip Size | 22 × 25 mm | 22 × 25 mm |

13.89 mg TE is equivalent to 10 mg testosterone base and 15.84 mg TU is equivalent to 10 mg testosterone base.

### Solvent Lamination of TE and TU Strips:

The films strips prepared as described above were then liminated to form bilayer film dosage composition. In particular, one TE strip was overlaid onto one TU strip by 2 mm and a 25% PVP/ethanol solution was coated between the overlaid films and let to dry at room temperature. The bilayer film is about 33 mm×22 mm and contains 20 mg of testosterone base.

### Pharmacokinetic Study

As part of the preclinical evaluation of these testosterone ester formulations, the pharmacokinetic profile of the testosterone enanthate/testosterone undecanoate bilayer film dosage composition was compared to the pharmacokinetic profile of FORTESTA® (testosterone gel) in minipigs.

Briefly, on Day 1, three (3) castrated Gottingen minipigs were anesthetized, the oral cavity was exposed and the testosterone enanthate/testosterone undecanoate bilayer film dosage composition was placed on the buccal mucosa of each pig with the testosterone enanthante layer in direct contact with the buccal mucosa. As discussed above, each testosterone enanthate/testosterone undecanoate bilayer film dosage composition contains the equivalent of 20 mg of testosterone base.

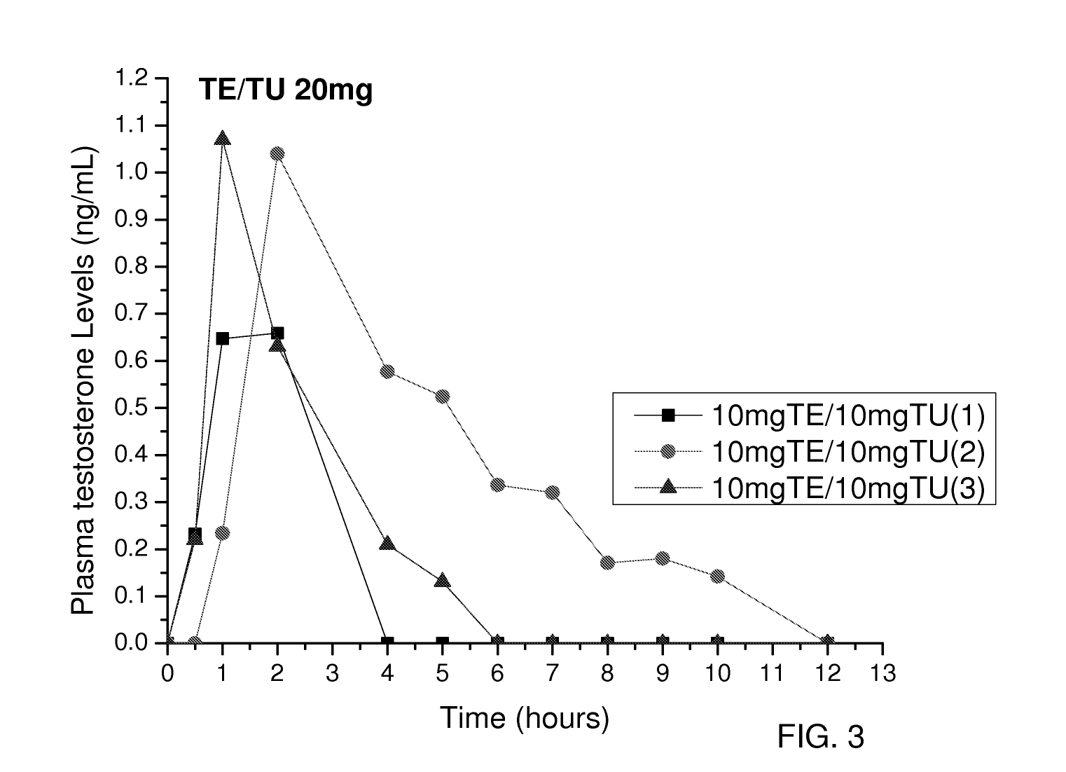

Blood samples were collected periodically over 12 hours and the plasma analyzed for testosterone using an HPLC-MS/MS analytical method. The pharmacokinetic profile of each pig is shown in FIG. 3.

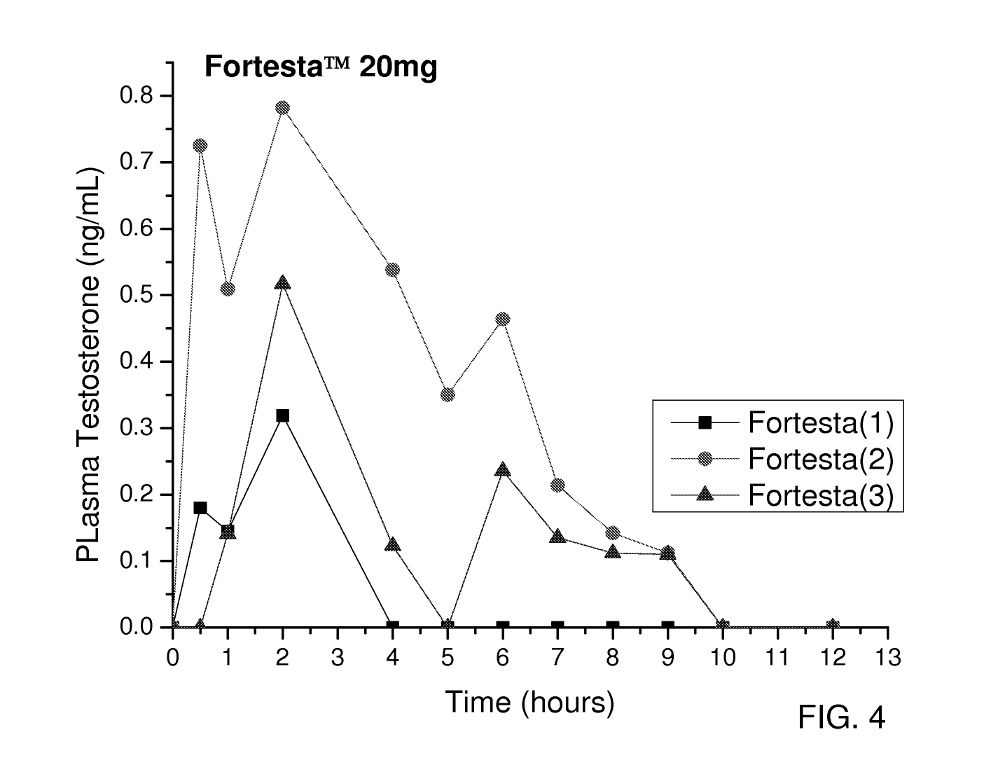

In a control study, the same three minipigs were dosed with 20 mg FORTESTA® (testosterone) topical gel. Blood samples were also collected periodically over 12 hours from each animal and the plasma analyzed for testosterone using an HPLC-MS/MS analytical method. The pharmacokinetic profile of each pig is shown in FIG. 4.

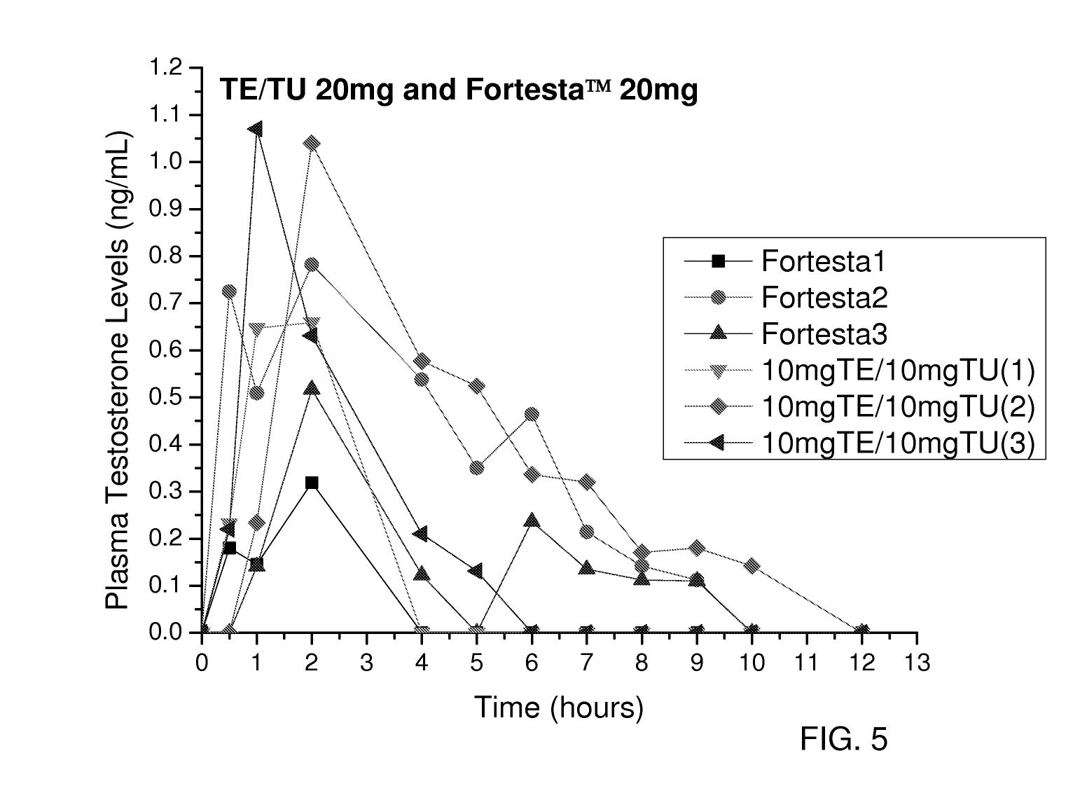

A comparison of the pharmacokinetic profiles of the buccal testosterone ester films to the pharmacokinetic profiles of topical FORTESTA® (testosterone) gel (FIG. 5) shows that both dosage forms provide sustained delivery of testosterone for a minimum of 8 hours. In fact, the film dosage compositions of the present invention provided a maximum plasma testosterone levels about 30% greater than that show from FORTESTA® in 2 instances.

### Secondary Construct

The secondary construct is prepared using the primary construct as an “ingredient” in formulating a dosage form suitable for administration. A skilled artisan of pharmaceutical formulations can readily adapt conventional techniques for formulating pharmaceutical dosage forms to employ a primary construct of the present invention as an ingredient therein. Importantly, the primary construct may be used in conjunction with the same active found in the primary construct or a different active. Alternatively, the primary construct may be the sole source of active in the dosage form. Also, additional additives may be employed to increase the stability of the steroid hormone delivery system.

Notably, a secondary construct in the form of a film can be prepared using the primary construct as an active ingredient during the preparation of the film. For example, a secondary construct in the form of a film can be prepared using the primary construct as one “ingredient” in the mixture that is used to cast PharmFilm® (MonoSol Rx, Warren, N.J.).

What is claimed is:

1. A film dosage composition comprising:
a. A first region comprising:
i. A first polymeric matrix; and
ii. A first plurality of particles comprising:
1. A first testosterone ester; and
2. A first surfactant; and
b. A second region comprising:
i. A second polymeric matrix; and
ii. A second plurality of particles comprising:
1. A second testosterone ester; and
2. A first surfactant.

2. The film dosage composition of claim 1, wherein said first testosterone ester and second testosterone ester are independently selected from the group consisting of testosterone enanthate, testosterone undecanoate, testosterone cypionate, testosterone propionate, testosterone formate, testosterone acetate, testosterone butyrate, testosterone valerate, testosterone caproate, testosterone isocaproate, testosterone heptanoate, testosterone octanoate, testosterone nonanoate, testosterone decanoate and combinations thereof.

3. The film dosage composition of claim 1, wherein said first testosterone ester is testosterone enanthate and said second testosterone ester is testosterone undecanoate.

4. The film dosage composition of claim 1, wherein said first surfactant and second surfactant are independently selected from the group consisting of ethoxy (35) castor oil, diethylene glycol monoethyl ether, propylene glycol monocaprylate, stearoyl macrogol-32 glycerides, stearoyl polyoxyl-32 glycerides, stearoyl polyoxylglycerides, sodium dodecylsulfate, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monooleate, sorbitan monostearate, sorbitan palmitate, sorbitan monolaurate, and combinations thereof.

5. The film dosage composition of claim 1, wherein said first surfactant is a combination of ethoxy (35) castor oil, diethylene glycol monoethyl ether, and propylene glycol monocaprylate and said second surfactant is stearoyl polyoxylglyceride.

6. The film dosage composition of claim 1, wherein said first plurality of particles and second plurality of particles are independently selected from the group consisting of an emulsion, micelles, liposomes, lipid particles, nanoparticles, niosomes, and combinations thereof.

7. The film dosage composition of claim 1, wherein said first plurality of particles and second plurality of particles are both microemulsions.

8. The film dosage composition of claim 1, wherein said first region is a first film and said second region is a second film.

9. The film dosage composition of claim 8, wherein said first film and said second film are laminated to one another.

10. The film dosage composition of claim 1, wherein said first polymeric matrix and said second polymeric matrix independently comprise a polymer selected from the group consisting of polyethylene oxide, cellulose, a cellulose derivative, pullulan, polyvinyl pyrrolidone, polyvinyl alcohol, polyethylene glycol, carboxyvinyl copolymers, hydroxypropylmethyl cellulose, hydroxyethyl cellulose, hydroxypropyl cellulose, carboxymethyl cellulose, sodium alginate, xanthan gum, tragacanth gum, guar gum, acacia gum, arabic gum, polyacrylic acid, methylmethacrylate copolymer, carboxyvinyl copolymers, starch, gelatin, ethylcellulose, cellulose acetate phthalate, hydroxypropyl methyl cellulose phthalate, polyvinylacetatephthalates, phthalated gelatin, crosslinked gelatin, poly(lactic acid)/poly(glycolic acid)/polyethyleneglycol copolymers, polycaprolactone, methylmethacrylate copolymer, polyacrylic acid polymer, poly(glycolic acid) (PGA), poly(lactic acid) (PLA), poly(lactic acid)/poly(glycolic acid)/polyethyleneglycol copolymers, polydioxanones, polyoxalates, poly(a-esters), polyanhydrides, polyacetates, polycaprolactones, poly(orthoesters), polyamino acids, polyaminocarbonates, polyurethanes, polycarbonates, polyamides, poly(alkyl cyanoacrylates), sodium alginate, xanthan gum, tragacanth gum, guar gum, acacia gum, arabic gum, starch, gelatin, carageenan, locust bean gum, dextran, gellan gum, and combinations thereof.

11. A film dosage composition comprising:
a. A first film comprising:
i. A first polymeric matrix comprising polyethylene oxide and hydroxypropylmethyl cellulose; and
ii. A first plurality of particles comprising:
1. Testosterone enanthate; and
2. A surfactant comprising a combination of ethoxy (35) castor oil, diethylene glycol monoethyl ether, and propylene glycol monocaprylate; and
b. A second film comprising:
i. A second polymeric matrix comprising polyethylene oxide and hydroxypropylmethyl cellulose; and
ii. A second plurality of particles comprising:
1. Testosterone undecanoate; and
2. A surfactant comprising stearoyl polyoxylglycerides
wherein said first film and said second film are laminated to one another.

12. A process of forming a film dosage composition comprising the steps of:
a. Forming a first admixture of a first testosterone ester and a first surfactant;
b. Combining said first admixture of a first testosterone ester and a first surfactant with a first polymeric matrix to produce a first film-forming composition comprising a first plurality of particles;
c. Casting said first film-forming composition to form a first film;
d. Forming a second admixture of a second testosterone ester and a second surfactant;
e. Combining said second admixture of a second testosterone ester and a second surfactant with a second polymeric matrix to produce a second film-forming composition comprising a second plurality of particles;
f. Casting said second film-forming composition to form a second film; and
g. Combining said first film and said second film to one another to form a film dosage composition.

13. The process of forming a film dosage composition 12, wherein said first testosterone ester and second testosterone ester are independently selected from the group consisting of testosterone enanthate, testosterone undecanoate, testosterone cypionate, testosterone propionate, testosterone formate, testosterone acetate, testosterone butyrate, testosterone valerate, testosterone caproate, testosterone isocaproate, testosterone heptanoate, testosterone octanoate, testosterone nonanoate, testosterone decanoate and combinations thereof.

14. The process of forming a film dosage composition 12, wherein said first testosterone ester is testosterone enanthate and said second testosterone ester is testosterone undecanoate.

15. The process of forming a film dosage composition 12, wherein said first surfactant and second surfactant are independently selected from the group consisting of ethoxy (35) castor oil, diethylene glycol monoethyl ether, propylene glycol monocaprylate, stearoyl macrogol-32 glycerides, stearoyl polyoxyl-32 glycerides, stearoyl polyoxylglycerides, sodium dodecylsulfate, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monooleate, sorbitan monostearate, sorbitan palmitate, sorbitan monolaurate, and combinations thereof.

16. The process of forming a film dosage composition 12, wherein said first surfactant is a combination of ethoxy (35) castor oil, diethylene glycol monoethyl ether, and propylene glycol monocaprylate and said second surfactant is stearoyl polyoxylglyceride.

17. The process of forming a film dosage composition 12, wherein said first plurality of particles and second plurality of particles are independently selected from the group consisting of an emulsion, micelles, liposomes, lipid particles, nanoparticles, niosomes, and combinations thereof.

18. The process of forming a film dosage composition 12, wherein said first plurality of particles and second plurality of particles are both microemulsions.

19. The process of forming a film dosage composition 12, wherein said first polymeric matrix and said second polymeric matrix independently comprise a polymer selected from the group consisting of polyethylene oxide, cellulose, a cellulose derivative, pullulan, polyvinyl pyrrolidone, polyvinyl alcohol, polyethylene glycol, carboxyvinyl copolymers, hydroxypropylmethyl cellulose, hydroxyethyl cellulose, hydroxypropyl cellulose, carboxymethyl cellulose, sodium alginate, xanthan gum, tragacanth gum, guar gum, acacia gum, arabic gum, polyacrylic acid, methylmethacrylate copolymer, carboxyvinyl copolymers, starch, gelatin, ethylcellulose, cellulose acetate phthalate, hydroxypropyl methyl cellulose phthalate, polyvinylacetatephthalates, phthalated gelatin, crosslinked gelatin, poly(lactic acid)/poly(glycolic acid)/polyethyleneglycol copolymers, polycaprolactone, methylmethacrylate copolymer, polyacrylic acid polymer, poly(glycolic acid) (PGA), poly(lactic acid) (PLA), poly(lactic acid)/poly(glycolic acid)/polyethyleneglycol copolymers, polydioxanones, polyoxalates, poly(a-esters), polyanhydrides, polyacetates, polycaprolactones, poly(orthoesters), polyamino acids, polyaminocarbonates, polyurethanes, polycarbonates, polyamides, poly(alkyl cyanoacrylates), sodium alginate, xanthan gum, tragacanth gum, guar gum, acacia gum, arabic gum, starch, gelatin, carageenan, locust bean gum, dextran, gellan gum, and combinations thereof.

20. The film dosage composition of claim 8, wherein said first film and said second film are co-extruded.

21. The film dosage composition of claim 8, wherein said first film and said second film are co-coated with a die-slot.

22. The process of claim 12, wherein the combining step comprises laminating said first film to said second film.

23. The process of claim 12, wherein the combining step comprises co-coating the first film and second film by a slot die process.
